(19) United States
(12) Patent Application Publication
(10) Pub. No.: US 2026/0240596 A1
(43) Pub. Date: Aug. 20, 2026

(54) ELECTROSURGICAL INSTRUMENT AND ELECTROSURGICAL APPARATUS

(71) Applicant: Creo Medical Limited, Chepstow (GB)

(72) Inventors: Christopher Paul HANCOCK, Chepstow (GB); George Christian ULLRICH, Chepstow (GB); Louis TURNER, Chepstow (GB); David Edward WEBB, Chepstow (GB); Steven THOMAS, Chepstow (GB); Warren JONES, Chepstow (GB); Duncan James Foster FITZSIMONS, St. Albans (GB)

(21) Appl. No.: 19/161,095

(22) PCT Filed: Feb. 29, 2024
(86) PCT No.: PCT/EP2024/055271

§ 371 Date: Aug. 29, 2025

(30) Foreign Application Priority Data
Mar. 3, 2023 (GB) 2303160.2
Mar. 3, 2023 (GB) 2303162.8
Jun. 19, 2023 (GB) 2309167.1

(51) Int. Cl. A61B 18/18; A61B 18/00; A61B 18/12; A61B 18/14

(52) U.S. Cl. CPC A61B 18/1815; A61B 18/1206; A61B 18/1445; A61B 2018/00077; A61B 2018/00083; A61B 2018/00178; A61B 2018/00184; A61B 2018/00601; A61B 2018/0063; A61B 2018/00982; A61B 2018/00994; A61B 2018/1455; A61B 2018/1823; A61B 2018/1861

(57) ABSTRACT

An electrosurgical instrument delivers both microwave and radiofrequency (RF) energy to selectively seal or cut tissue. A coaxial or other microwave/RF transmission line terminates at a distal jaw assembly having opposed first and second jaws movable between open and closed positions for tissue capture. The jaws carry first and second pairs of electrodes. The first pair, spaced apart on confronting jaw surfaces, is coupled to the microwave feed to define a microwave sealing zone that effects rapid dielectric heating and vessel fusion. The second pair is coupled to the RF feed to act, respectively, as active and return electrodes, defining a laterally offset RF cutting zone that generates concentrated current density for precise tissue division. Physical separation of the sealing and cutting zones facilitates sequential or simultaneous hemostasis and transection using a single, compact instrument.

### FIELD OF THE INVENTION

The invention relates to an electrosurgical instrument for sealing and/or cutting tissue, using delivery of electromagnetic energy to tissue. The instrument comprises a pair of jaws between which tissue can be gripped, the jaws including electrodes for delivery of energy to the tissue.

### BACKGROUND TO THE INVENTION

Electrosurgical instruments for delivering heat energy into grasped biological tissue are known. For example, it is known to deliver microwave energy from a bipolar electrode arrangement in the jaws of a forceps. The microwave energy may be used to seal a vessel by thermal denaturation of extracellular matrix proteins (e.g. collagen) within the vessel wall. The heat energy may also cauterise the grasped tissue and facilitate coagulation.

It is also known to use radiofrequency (RF) energy for cutting tissue. Tissue cutting using RF energy operates based on the principle that as an electric current passes through a tissue matrix (aided by the ionic contents of the cells), the impedance to the flow of electrons across the tissue generates heat. When a pure sine wave is applied to the tissue matrix, enough heat is generated within the cells to vaporise the water content of the tissue. There is thus a large rise in the internal pressure of the cell that cannot be controlled by the cell membrane, resulting in the cell rupturing. When this occurs over a wide area it can be seen that tissue has been transected.

Such electrosurgical instruments typically find application on the end of minimally invasive surgical laparoscopic tools but can equally find use in other clinical procedural areas such as gynaecology, endourology, gastrointestinal surgery, ENT procedures, or endoscopic procedures. Depending on the context of use, these devices can have differing physical construction, size, scale and complexity.

For example, a gastrointestinal instrument might be nominally of 3 mm diameter mounted on to the end of a very long flexible shaft. In contrast, a laparoscopic instrument may be used on the end of an industry standard nominal 5 mm or 10 mm diameter rigid or steerable steel shaft.

U.S. Pat. No. 6,585,735 describes an endoscopic bipolar forceps in which the jaws of the forceps are arranged to conduct bipolar energy through the tissue held therebetween.

EP 2 233 098 describes microwave forceps for sealing tissue in which the sealing surfaces of the jaws include one or more microwave antennas for radiating microwave energy into tissue grasped between the jaws of the forceps.

WO 2015/097472 describes electrosurgical forceps in which one or more pairs of non-resonant unbalanced lossy transmission line structures are arranged on the inner surface of a pair of jaws.

### SUMMARY OF THE INVENTION

At its most general, the present invention provides various types of electrosurgical instruments that can enable fine tissue cutting and dissection to be performed on tissue.

Moreover, the electrosurgical instruments may provide additional functionality, such as sealing biological tissue, such as (blood) vessels, using a confined microwave field that can yield a well-defined seal location with low thermal margin. With these additional functions, fewer device interchanges may be needed during a procedure.

The electrosurgical instruments disclosed herein may be used in any type of surgical procedure, but it is expected to find particular utility for non-invasive or minimally invasive procedures. For example, the electrosurgical instruments may be configured to be introduced to a treatment site through an instrument channel of a surgical scoping device, such as a laparoscope or an endoscope.

According to a first aspect of the invention, there is provided an electrosurgical instrument for sealing and/or cutting tissue, comprising: a transmission line for conveying microwave electromagnetic energy and/or radiofrequency electromagnetic energy; a pair of jaws comprising a first jaw and a second jaw mounted at a distal end of the transmission line, wherein the first jaw and the second jaw are movable between an open position, in which tissue can be inserted into a gap between the first jaw and the second jaw, and a closed position, in which the first jaw and the second jaw are brought together to clamp tissue therebetween; wherein the pair of jaws comprises a first pair of electrodes comprising a first electrode and a second electrode, and a second pair of electrodes comprising a third electrode and a fourth electrode; wherein the first pair of electrodes is configured to receive microwave energy conveyed by the transmission line and to emit the received microwave energy into tissue located between the first jaw and the second jaw, such that a microwave sealing area is defined on an inner surface of the pair of jaws between the first and second electrodes; wherein the second pair of electrodes is configured to receive radiofrequency energy conveyed by the transmission line and to operate as active and return electrodes for delivering the radiofrequency energy to tissue located between the first jaw and the second jaw, such that a radiofrequency cutting area is defined on an inner surface of the pair of jaws between the second and third electrodes; and wherein the first pair of electrodes and the second pair of electrodes are arranged such that, when the first jaw and the second jaw are in the closed position, the microwave sealing area is spaced apart from the radiofrequency cutting area in a lateral direction normal to a longitudinal axis of the pair of jaws.

The electrosurgical instrument of the invention may be used to perform vessel sealing and vessel dividing. Vessel sealing can be performed by clamping a vessel between the pair of jaws to squash the walls of the vessel, followed by application of microwave energy to the vessel via the first pair of electrodes. Application of microwave energy to the vessel may result in dielectric heating of the gripped tissue, which may cause the tissue cells to be disrupted/denatured and form an amalgam of collagen predominant in vessel walls, which effectively bonds the vessel walls together. With time, post operatively, cellular recovery and regrowth occurs to reinforce the seal further. Vessel dividing is a process of cutting through a continuous biological vessel to separate it into two pieces. It is normally performed after a vessel is first sealed, e.g. via delivery of microwave energy as described above. With the electrosurgical instrument of the invention, vessel dividing may be performed by delivery of radiofrequency (RF) energy via the second pair of electrodes to tissue clamped between the jaws. Delivery of RF energy to the tissue causes the vessel to be cut (resected) along the second pair of electrodes.

The inventors have found that the process of sealing tissue using microwave energy causes the tissue to become charred and dried out. However, if tissue becomes too dry, it becomes difficult to effectively cut the tissue using RF energy, as the tissue may not be sufficiently conductive. As a result, it may be difficult to cut tissue in an area which has been sealed with microwave energy. The electrosurgical instrument of the invention addresses this issue by providing dedicated pairs of electrodes for performing microwave sealing and RF cutting, where a lateral spacing is provided between the two pairs of electrodes. In this manner, the RF cutting may be performed on a portion of tissue which is laterally spaced from (e.g. adjacent to) a portion of tissue which was sealed via microwave energy with the first pair of electrodes. The lateral spacing between the RF electrodes and the microwave electrodes may serve to ensure that the portion of tissue on which RF cutting is performed is not completely dried out, such that an effective RF cut can be performed. Moreover, the lateral spacing between the RF and microwave electrodes may avoid interference between the RF and microwave fields, which may facilitate simultaneous delivery of microwave and RF energy.

Thus, the electrosurgical instrument of the invention may enable effective sealing and cutting of a vessel. In particular, a vessel can be clamped between the jaws and sealed using microwave energy delivered via the first pair of electrodes. Then, keeping the vessel clamped in the jaws, the vessel can be cut at a location adjacent to the seal using RF energy delivered via the second pair of electrodes. The provision of a microwave sealing area and an RF cutting area on an inner surface of the jaws enables a single instrument to be used for performing both sealing and resection of tissue without having to move or reposition the instrument between the sealing and cutting procedures.

The transmission line may comprise any suitable energy conveying structure for conveying microwave electromagnetic energy and RF electromagnetic energy to the pair of jaws.

The microwave electromagnetic energy and the RF electromagnetic energy may be conveyed along a common signal pathway. For example, the transmission line may comprise a coaxial cable that provides the common signal pathway for conveying both the microwave energy and the radiofrequency energy. In this arrangement, the transmission line may comprise a first filter (e.g. an inductive filter) for blocking the microwave energy from reaching the second pair of electrodes, and a second filter (e.g. a capacitive filter) for blocking the RF energy from reaching the first pair of electrodes.

In an alternative arrangement, the radiofrequency energy and microwave energy are conveyed along separate pathways. In other words, the transmission line may include separate pathways for conveying the RF and microwave energy, respectively. For example, the transmission line may comprise a coaxial cable for conveying the microwave electromagnetic energy, and two or more wires for conveying the RF electromagnetic energy. In such a case, the first filter and the second filter may be provided at a proximal end of the transmission line, e.g. in a handle.

The electrosurgical instrument may further comprise an instrument shaft through which the transmission line extends. The instrument shaft may be dimensioned to fit within an instrument channel of a surgical scoping device. The surgical scoping device may be a laparoscope or an endoscope. Surgical scoping devices are typically provided with an insertion tube that is a rigid or flexible (e.g. steerable) conduit that is introduced into a patient's body during an invasive procedure. The insertion tube may include the instrument channel and an optical channel (e.g. for transmitting light to illuminate and/or capture images of a treatment site at the distal end of the insertion tube). The instrument channel may have a diameter suitable for receiving invasive surgical tools. The diameter of the instrument channel may be equal to or less than 13 mm, preferably equal to or less than 10 mm, and more preferably, especially for flexible insertion tubes, equal to or less than 5 mm.

The transmission line may be flexible to facilitate insertion into the instrument channel of the scoping device. Likewise, the instrument shaft may be flexible. Further, the transmission line may be arranged within a lumen of the shaft. The instrument shaft may cover and/or shield the transmission line. The transmission line may extend from a distal end to a proximal end of the electrosurgical instrument. In particular, the transmission line may electrically connect the first and second pairs of electrodes to an electrosurgical generator arranged to generate the microwave and/or RF energy.

The first jaw and/or the second jaw may be movable relative to the distal end of the transmission line. The first jaw and/or the second jaw may be attached to the distal end of the transmission line, and/or a distal end of the instrument shaft, via a joint (or hinge). The joint may include a pivot axis around which the first jaw and/or the second jaw may rotate. The first jaw and/or the second jaw may be activated by one or more actuation rods or control wires respectively connected to the first jaw and/or the second jaw. The one or more actuation rods or control wires may extend within the instrument shaft to a proximal end of the electrosurgical instrument. The one or more actuation rods may be connected to a handle with which the first and/or second jaws can be actuated, e.g. opened and/or closed. The electrosurgical instrument may comprise an actuation mechanism which converts a back-and-forth movement of the actuation rod(s) or control wire(s) into a rotational movement of the first jaw and/or the second jaw.

For example, both jaws can be movable, e.g. rotatable around a (common) pivot axle. In another embodiment, one of the jaws is fixed to the shaft and the other jaw is movable relative to the one jaw.

In the open position, the first jaw and the second jaw are (maximally) spaced apart so that there is a free space (gap) between inner surfaces of the two jaws. In this way, tissue can be inserted between the jaws in the open position. Usually, the first jaw and the second jaw are moved towards the tissue such that the tissue is pushed into the space between the inner surfaces of the jaws in the open position of the first jaw and the second jaw.

By moving the first jaw and/or the second jaw from the open position to the closed position, the tissue between the first surface and the second surface can be grasped and/or clamped between the first jaw and the second jaw. In this way, the tissue can be fixed between the inner surfaces of the first and second jaws in the closed position.

The inner surface of the first jaw and the inner surface of the second jaw correspond to faces (surfaces) of the first jaw and the second jaw, respectively, that face towards each other in the open and/or closed position. In the closed position, the inner surfaces of the two jaws may extend parallel to one another. In the closed position, the inner surfaces of the two jaws may be in contact with one another.

The pair of jaws may be pivotable relative to each other about a hinge axis that lies transverse to a longitudinal axis of the transmission line (which may be the same as the longitudinal axis of the pair of jaws). In one example, the pair of jaws comprises a static jaw that is fixed relative to the instrument shaft, and a movable jaw that is pivotably mounted relative to the static jaw to open and close the gap between the opposing inner surfaces. In another example, both jaws are arranged to pivot with respect to the instrument shaft, e.g. in a symmetrical forceps-type or scissors-type arrangement.

In another example, the pair of jaws may be arranged to move relative to one another in a manner that maintains the inner surfaces thereof in an aligned, e.g. parallel, orientation. This configuration may be desirable for maintaining a uniform pressure on grasped tissue along the length of the jaws. One example of such a closure mechanism is disclosed in WO 2015/097472.

The first jaw and/or the second jaw may have a Maryland configuration. This can include that the first jaw and the second jaw are not straight but bent/curved, e.g. forming an arc or an S-shape in a side view.

The first pair of electrodes is configured to receive microwave energy from the transmission line and to emit (radiate) the received microwave energy. For example, where the transmission line comprises a coaxial cable, the first electrode may be connected to an inner conductor of the coaxial cable, and the second electrode may be connected to an outer conductor of the coaxial cable. The first pair of electrodes may form a dipole antenna for radiating microwave energy into target tissue.

The first pair of electrodes may be located in the first jaw or the second jaw. In other words, both the first and second electrodes may be located on the same jaw.

The first pair of electrodes is arranged such that microwave energy is emitted from an inner surface of the pair of jaws, such that the microwave energy is emitted into tissue that is in contact with the inner surface. In this manner, a microwave sealing area is defined on the inner surface of the jaws between the first and second electrodes, such that tissue in contact with the microwave sealing area will receive microwave energy emitted by the first pair of electrodes, e.g. so that the tissue can be sealed. The inner surface on which the microwave sealing area is defined may be on the first jaw or the second jaw, depending on which jaw the first pair of electrodes is located.

The first pair of electrodes may be exposed at the inner surface of one of the jaws. In this manner, the microwave sealing area may correspond to an area of the inner surface located between exposed portions of the first and second electrodes.

Alternatively, the first pair of electrodes may be partially or completely covered. For example, the first pair of electrodes may be partially or completely covered with an electrically insulating material. In such a case, the microwave sealing area may correspond to an area of the inner surface arranged between the first and second electrodes.

The second pair of electrodes is configured to receive RF energy from the transmission line and deliver the RF energy to tissue located between the jaws. For example, the third electrode may be connected to the transmission line to act as an active electrode and the fourth electrode may be connected to the transmission line to act as a return electrode for the RF energy (or vice versa).

The second pair of electrodes may be located in the first jaw or the second jaw. In other words, both the third and fourth electrodes may be located on the same jaw.

The second pair of electrodes may be exposed on the inner surface of one of the jaws, to enable delivery of the RF energy to tissue in contact with the inner surface of the jaw. In line with the discussion above, delivery of the RF energy to tissue via the second pair of electrodes may result in cutting of the tissue along the second pair of electrodes. Thus, an RF cutting area is defined on the inner surface of the jaw between the third electrode and the fourth electrode. The inner surface on which the RF cutting area is defined may be on the first jaw or the second jaw, depending on which jaw the second pair of electrodes is located.

The first pair of electrodes and the second pair of electrodes are separate (distinct) from one another, i.e. no electrodes are shared between the first and second pairs. This serves to avoid any overlap between the microwave sealing area and RF cutting area.

The first pair of electrodes and the second pair of electrodes are arranged in the jaws such that they are spaced in the lateral direction from one another. Thus, when the jaws are in the closed position, the second pair of electrodes may be offset from the first pair of electrodes in the lateral direction. As a result, the microwave sealing area is spaced (offset) in the lateral direction from the RF cutting area. In other words, the first pair of electrodes is arranged such that the microwave sealing area (between the first and second electrodes) and the RF cutting area (between the third and fourth electrodes) do not overlap (or overlie) one another when the jaws are in the closed position. In line with the discussion above, the lateral offset between the microwave sealing area and the RF cutting area may ensure that tissue sealing and cutting are performed at adjacent locations on a piece of tissue clamped between the jaws, thus facilitating RF cutting of the tissue following a microwave seal.

Herein, the longitudinal axis of the pair of jaws may correspond to a central axis of the pair of jaws which extends away from a proximal end of the pair of jaws (i.e. the end of the jaws located towards the distal end of the transmission line), towards the distal end of the pair of jaws. The longitudinal axis of the pair of jaws may correspond to a longitudinal axis of the transmission line. The lateral direction is perpendicular to the longitudinal axis, e.g. the lateral direction corresponds to a width direction of the jaws. The lateral direction may be substantially parallel to an inner surface of one or both of the jaws.

In some embodiments, the first pair of electrodes and the second pair of electrodes may be on the same jaw, e.g. on the first jaw. In such a case, the first pair of electrodes may be spaced apart from the second pair of electrodes in the lateral direction by a dielectric material in the first jaw. Then, both the microwave sealing area and the RF cutting area may be located on the inner surface of the first jaw, wherein the microwave sealing area and the RF cutting area are spatially separated from one another (i.e. they do not overlap).

Alternatively, the first pair of electrodes and the second pair of electrodes may be on different jaws, e.g. the first pair of electrodes may be on the first jaw and the second pair of electrodes may be on the second jaw. Then, the microwave sealing area may be on the inner surface of the first jaw and the RF cutting area may be on the inner surface of the second jaw. As discussed above, the microwave sealing area and the RF cutting area are then arranged such that they do not overlap one another when the jaws are in the closed position. In other words, when the jaws are in the closed position, a projection of the RF cutting area onto the first jaw along a direction normal to the inner surface of the first jaw does not overlap the microwave sealing area.

The first pair of electrodes and the second pair of electrodes may extend in a longitudinal direction of the jaws, e.g. in a direction substantially parallel to the longitudinal axis. Accordingly, the microwave sealing area may comprise an elongate region on the inner surface of the jaws, e.g. to create a correspondingly shaped tissue seal. Likewise, the RF cutting area may comprise an elongate region on the inner surface of the jaws, e.g. to create a correspondingly shaped cut in the tissue.

The RF cutting area may be substantially centred relative to the longitudinal axis. In other words, the third electrode and the fourth electrode may be substantially evenly spaced about the longitudinal axis in the lateral direction. In this manner, the RF cutting area may be centred with respect to a width of the pair of jaws. This may result in an RF cut towards the middle of the jaws, which may facilitate accurate cutting of tissue.

The first pair of electrodes may be shaped to define a first microwave sealing area and a second microwave sealing area, and the first and second microwave sealing areas may be arranged on either side of the second pair of electrodes when the first jaw and the second jaw are in the closed position. In other words, the microwave sealing area discussed above may comprise first and second microwave sealing areas. In this manner, when microwave energy is conveyed to the first pair of electrodes, tissue seals may be formed at the microwave sealing areas on either side of the second pair of electrodes. Then, RF cutting may be performed with the second pair of electrodes at a location between the two seals. This arrangement may facilitate vessel sealing and cutting, as sealing the vessel on either side of the RF cutting area may serve to ensure that both ends of the vessel are sealed when the vessel is cut in two. The first and second microwave sealing areas may be located towards an outside of the jaws, whilst the RF cutting area may be arranged towards a middle of the jaws.

The first microwave sealing area may be arranged such that it is spaced (offset) in the lateral direction on a first side from the RF cutting area, with the second microwave sealing area being spaced (offset) in the lateral direction on a second side from the RF cutting area. In this manner, when the jaws are in the closed position, the RF cutting area may be located between the first and second microwave sealing areas.

The first microwave sealing area may be defined between a first portion of the first and second electrodes laterally spaced on a first side from the second pair of electrodes, and the second microwave sealing area may be defined between a second portion of the first and second electrodes laterally spaced on a second side from the second pair of electrodes.

Where both the first and second pairs of electrodes are located on the first jaw, the RF cutting area may be located between the first and second microwave sealing areas on the inner surface of the first jaw. The RF cutting area may be separated from the first and second sealing areas by a dielectric material in the first jaw.

The first microwave sealing area and the second microwave sealing area may be substantially evenly spaced about the RF cutting area in the lateral direction (when the jaws are in the closed position). In other words, a first distance between the RF cutting area and the first microwave sealing area and a second distance between the RF cutting area and the second microwave sealing area may be equal. In this manner, the microwave seals may be formed evenly (e.g. symmetrically) on either side of the RF cutting area. This may serve to ensure that there is adequate spacing between the RF cutting area and the microwave sealing areas to facilitate RF cutting, as well as facilitate accurate sealing and cutting of tissue. When the RF cutting area is substantially centred relative to the longitudinal axis, then the first and second microwave sealing areas may likewise be substantially evenly spaced about the longitudinal axis in the lateral direction.

The first electrode may comprise a first side portion and a second side portion which are spaced in the lateral direction by a first gap, and wherein the first and second side portions of the first electrode are located on either side of the second pair of electrodes when the first jaw and the second jaw are in the closed position. Thus, the first portion of the first electrode may serve to define the first microwave sealing area, whilst the second portion of the first electrode may serve to define the second microwave sealing area. The first and second side portions of the first electrode may be electrically connected together, e.g. they may both be connected to the inner conductor of the coaxial cable. A width of the first gap may be greater than a width of the RF cutting area (in the lateral direction). This may ensure that the first and second microwave sealing areas are laterally spaced from the RF cutting area.

Where the first microwave sealing area and the second microwave sealing area are substantially evenly spaced about the radiofrequency cutting area in the lateral direction, the first and second side portions of the first electrode may be substantially evenly spaced about the longitudinal axis in the lateral direction.

Where the first and second pair of electrodes are in the first jaw, the first and second side portions of the first electrode may define a first channel therebetween, with the second pair of electrodes being located in the first channel. In other words, the first and second side portions of the first electrode may define sidewalls of the first channel in which the second pair of electrodes is located. The second pair of electrodes may be isolated and spaced apart from the first and second side portions by a dielectric material in the first channel.

The first electrode may further comprise a first connecting portion which connects the first and second side portions of the first electrode across the first gap. In this manner, the first connecting portion may provide a structural and electrical connection between the first and second side portions, which may improve a performance of the first electrode. The first connecting portion may extend along a length of the first and second side portions of the first electrode. Thus, for example, a length of the first electrode may have a U-shaped cross-section. The first connecting portion may define a bottom wall of the first channel mentioned above.

The second electrode may comprise a first side portion and a second side portion which are spaced in the lateral direction by a second gap, and wherein the first electrode is located within the second gap. In this manner, the first microwave sealing area may be defined between the first side portions of the first and second electrodes, and the second microwave sealing area may be defined between the second side portions of the first and second electrodes. The first and second side portions of the second electrode may be electrically connected together, e.g. they may both be connected to the outer conductor of the coaxial cable. The second gap may be larger than a width of the first electrode, so that the first electrode (including its spaced first and second portions) can fit in the second gap. Accordingly, the first electrode may be located between the side portions of the second electrode, whilst the second pair of electrodes may be located between the side portions of the first electrode. This arrangement may serve to provide localised microwave sealing areas around an outside of the RF cutting area.

The first and second side portions of the second electrode may define a second channel therebetween, with the first electrode being located in the second channel. In other words, the first and second side portions of the second electrode may define sidewalls of the second channel in which the first electrode is located. The first electrode may be isolated and spaced apart from the first and second side portions of the second electrode by a dielectric material in the second channel.

The second electrode may further comprise a second connecting portion which connects the first and second side portions of the second electrode across the second gap. In this manner, the second connecting portion may provide a structural and electrical connection between the first and second side portions of the second electrode, which may improve a performance of the second electrode. The second connecting portion may extend along a length of the first and second side portions of the second electrode. Thus, for example, a length of the second electrode may have a U-shaped cross-section. The second connecting portion may define a bottom wall of the second channel mentioned above. In this manner, the second electrode may partially surround the first electrode. This may serve to ensure that microwave energy is only at the inner surface of the jaws, i.e. the second electrode may act as a shield which blocks microwave energy from being emitted away from the inner surface of the jaws.

The second electrode may be formed by a conductive outer shell of one of the jaws. For example, where the first pair of electrodes is in the first jaw, the second electrode may be formed by a conductive outer shell of the first jaw.

Where the first microwave sealing area and the second microwave sealing area are substantially evenly spaced about the radiofrequency cutting area in the lateral direction, the first and second side portions of the second electrode may be substantially evenly spaced about the longitudinal axis in the lateral direction. In particular, the first and second sides of the both the first and second electrodes may be substantially evenly spaced about the RF cutting area, to provide first and second microwave sealing areas that are evenly spaced about the RF cutting area.

Where the first jaw comprises the first pair of electrodes and the second pair of electrodes, the first pair of electrodes may be spaced apart from the second pair of electrodes by a dielectric material in the first jaw. Thus, the dielectric material may be disposed between the first pair of electrodes and the second pair of electrodes to isolate the two pairs of electrodes from one another. The dielectric material may comprise any suitable electrically insulating (isolating) material. A portion of the dielectric material may be exposed at the inner surface of the first jaw between the first and second pairs of electrodes, e.g. between the first electrode and the second pair of electrodes. The first electrode and the second electrode may be separated by a dielectric material, which may be the same or different from the dielectric material separating the first and second pairs of electrodes. Likewise, the third and fourth electrodes may be separated by a dielectric material, which may be the same or different from the dielectric material separating the first and second pairs of electrodes.

The second jaw may comprise a third pair of electrodes comprising a fifth electrode and a sixth electrode. The third pair of electrodes may be configured to receive microwave energy conveyed by the transmission line and to emit the received microwave energy into tissue located between the first jaw and the second jaw, such that a microwave sealing area is defined on an inner surface of the second jaw between the fifth and sixth electrodes. The third pair of electrodes may be arranged such that, when the first jaw and the second jaw are in the closed position, the microwave sealing area on the inner surface of the second jaw is spaced apart from the radiofrequency cutting area in the lateral direction. In this manner, microwave sealing areas may be provided on the inner surface of the first jaw (between the first and second electrodes) and on the inner surface of the second jaw (between the fifth and sixth electrodes). Thus, when a piece of tissue is clamped between the jaws, microwave energy can be applied to both sides of the piece of tissue, which may facilitate sealing the tissue through its entire thickness. In line with the discussion above, as the microwave sealing area on the second jaw is laterally offset from the RF cutting area, the portion of tissue on which RF cutting is performed may not be entirely dried out from the microwave sealing.

The third pair of electrodes may be connected to the transmission line in a similar manner to the first pair of electrodes discussed above, e.g. the fifth electrode may be connected to the inner conductor of the coaxial cable and the sixth electrode may be connected to the outer conductor of the coaxial cable (or vice versa).

The third pair of electrodes may be arranged such that the microwave sealing area on the inner surface of the second jaw overlies (i.e. is aligned with) the microwave sealing area on the inner surface of the first jaw, when the jaws are in the closed position. In this manner, the sealing areas on the first and second jaws may cooperate to seal a piece of tissue from opposing sides, which may enhance a quality of the seal.

The third pair of electrodes may have a structure that mirrors a structure of the first pair of electrodes (e.g. when the jaws are in the closed position). In other words, the layout of the third pair of electrodes in the second jaw may be a mirror image of the first pair of electrodes in the first jaw. This may serve to ensure a symmetry of the microwave seals performed on tissue clamped between the jaws, to provide a well-defined and localised seal. Accordingly, any of the features discussed above in relation to the first pair of electrodes are equally applicable to the third pair of electrodes. For example, the third pair of electrodes may be shaped to define third and fourth microwave sealing areas which are arranged on either side of the RF cutting area when the jaws are in the closed position. The fifth electrode may have first and second side portions, and optionally a connecting portion. Likewise, the sixth electrode may have first and second side portions between which the fifth electrode is located, and optionally a connecting portion. The sixth electrode may be formed by a conductive outer shell of the second jaw.

A minimum spacing in the lateral direction between the microwave sealing area and the RF cutting area may be at least0.1 mm. The inventors have found that such a spacing may be sufficient to ensure that, when tissue is clamped between the jaws, tissue in contact with the RF cutting area does not become completely dried out by microwave energy applied to the tissue at the microwave sealing area, such that RF cutting may be effectively performed with the second pair of electrodes.

Increasing the lateral spacing between the microwave sealing area and the RF cutting area may reduce tissue drying in the RF cutting area due to application of microwave energy at the microwave sealing area, thus improving a quality of the RF cut. Thus, in some cases, the minimum spacing in the lateral direction between the microwave sealing area and the RF cutting area may be at least 0.3 mm, or at least 0.4 mm, or at least 0.5 mm.

A minimum spacing in the lateral direction between the first electrode and the second electrode may, for example, be at least 0.1 mm. In some cases, the minimum spacing in the lateral direction between the first electrode and the second electrode may be between 0.1 mm and 3 mm. The spacing in the lateral direction between the first electrode and the second electrode may correspond to a width of the microwave sealing area.

A minimum spacing in the lateral direction between the third electrode and the fourth electrode may, for example, be at least 0.1 mm. In some cases, the minimum spacing in the lateral direction between the third electrode and the fourth electrode may be between 0.1 mm and 3 mm. The spacing in the lateral direction between the third electrode and the fourth electrode may correspond to a width of the RF cutting area.

The second pair of electrodes may protrude from an inner surface of a first one of the first and second jaws. In other words, the second pair of electrodes may include a portion that sticks out from the inner surface of the jaws. In this manner, when tissue is clamped between the jaws, the protruding second pair of electrodes may be pressed into the tissue, which may serve to enhance a quality of RF cutting performed with the second pair of electrodes. This may also improve an evenness of the RF cut, by ensuring that the tissue is in close contact with the second pair of electrodes along a length of the second pair of electrodes. Where the second pair of electrodes is in the first (second) jaw, they may protrude from the inner surface of the first (second) jaw.

A second one of the first and second jaws may comprise a recess for receiving a protruding portion of the second pair of electrodes when the first and second jaw are in the closed position. This may facilitate bringing the jaws to the closed position when tissue is located between the jaws. Additionally, when tissue is located between the jaws, a portion of the tissue may be firmly held in the recess by the protruding second pair of electrodes, which may contribute to providing a well-defined and localised cut along tissue held in the recess. The recess may be formed in the inner surface of the jaw, and shaped to receive the protruding portion of the second pair of electrodes. For example, the recess may be in the form of a channel or groove in the inner surface of the jaw. Where the second pair of electrodes is in the first jaw, then the recess may be in the second jaw (or vice versa).

Ends of the third and fourth electrodes may be exposed at a distal end of a first one of the first and second jaws. In this manner, the ends of the third and fourth electrodes that are exposed on the distal end can act as an active electrode and a return electrode for RF cutting. This may allow fine cutting at the distal end of the electrosurgical instrument, for example, for cutting a hole into tissue located in front of the instrument so that the instrument can be further advanced into and/or through the tissue, for example, as part of a tunnelling procedure. Further, the third and fourth electrodes exposed on the distal end can be used to cut fine or small sections of tissue that are clamped or grasped between the jaws. Thus, tissue can be “nibbled”. As an example, where the second pair of electrodes is in the first jaw, the ends of the third and fourth electrodes may be exposed at a distal end (e.g. distal end face) of the first jaw.

The ends of the third and fourth electrodes may be exposed on a distal end face (surface) of the jaws, where the distal end face faces in a distal (forward) direction, i.e. away from the transmission line. In this manner, the exposed ends of the third and fourth electrodes can be used to deliver RF energy to tissue located in front of the jaws.

A second one of the first and second jaws may comprise an overhang portion arranged to cover the exposed ends of the third and fourth electrodes when the first and second jaws are in the closed position. This may enable tissue to be clamped or grasped between the distal end and the overhang portion of the jaws. In this manner, small portions of tissue clamped between the distal end and the overhang portion can be cut with RF energy applied to the second pair of electrodes. This may enable fine cutting of tissue over a relatively small area at the distal end of the jaw, e.g. compared to the larger RF cutting area located on the inner surface of the jaws.

A slot may be defined between the third electrode and the fourth electrode, and the instrument may further comprise a blade which is movable along the slot to cut tissue located between the first jaw and the second jaw. In this manner, the instrument may enable mechanical cutting with the blade, in addition to RF cutting.

As the slot is defined between the third electrode and the fourth electrode, the blade may move along the RF cutting area. In this manner, the blade may complement RF cutting, by facilitating separation (cutting) of tissue along the RF cutting area. The blade may be electrically isolated from the third and fourth electrodes. Alternatively, the blade may be electrically connected to one of the third and fourth electrodes, e.g. so that the blade can act as an active electrode for delivering RF energy to tissue in the RF cutting area.

The electrosurgical instrument may include any suitable mechanism for moving the blade along the slot. For example, the instrument may comprise a control wire or an actuation rod for actuating the blade. The control wire or actuation rod may extend within the instrument shaft and be connected to a handle.

The second pair of electrodes may be supported in one of the jaws by an isolating portion comprising an elastically deformable material, such that the second pair of electrodes may be movable in the jaw upon application of pressure to the second pair of electrodes. The second pair of electrodes can be considered as floating in the isolating portion. In other words, the second pair of electrodes can be solely attached to the jaw via the isolating portion. For instance, there may be no structural parts which support the second pair of electrodes in the jaw, except for the isolating portion. As an example, the isolating portion may support the second pair of electrodes relative to a rigid outer shell of the jaw in which the second pair of electrodes is located. The rigid outer shell may, for example, correspond to the conductive outer shell of the jaw mentioned above.

A flexible electrical connection (e.g. flexible wires) may provide the electrical connections between the second pair of electrodes and the transmission line. So, the pair of electrodes is movable relative to other components of the jaw. For example, where the first and second pairs of electrodes are in the first jaw, the second pair of electrodes may be movable relative to the first pair of electrodes. The movement of the second pair of electrodes is associated with a deformation (e.g. compression or stretching) of the elastically deformable material in the isolating portion. For example, if a pressure is applied to the second pair of electrodes (i.e. to the third and/or fourth electrode) so that the second pair of electrodes is pressed into the isolating portion (e.g. when tissue is grasped between the first jaw and the second jaw), the isolating portion may be compressed and the second pair of electrodes may move away from the opposing jaw and/or relative to the first pair of electrodes.

The ability of the second pair of electrodes to move in response to compression may an evenness of pressure on the tissue along the RF cutting area, and avoid pinch points. This may be particularly relevant if the second pair of electrodes protrudes from the inner surface of the jaw, such that the pressure between the second pair of electrodes and the opposing jaw may be higher compared other regions of the inner surfaces of the jaws. Additionally, the compression of the isolating portion provides a force further compressing the tissue between the second pair of electrodes and the opposing jaw, which may be helpful for RF cutting.

The elastically deformable material may comprise any electrically insulating material that is elastically deformable in response to application of pressure to the second pair of electrodes. The elastically deformable material may have a greater flexibility (i.e. be more readily deformable) than the second pair of electrodes or a structure on which the second pair of electrodes is formed. The elastically deformable material may also have a greater flexibility than rigid outer shells of the jaws.

Where the first pair of electrodes and the second pair of electrodes are in the first jaw, the isolating portion may correspond to the dielectric material mentioned above. In other words, the first and second pairs of electrodes may be spaced apart by the isolating portion. The elastically deformable material may then allow some relative movement between the second pair of electrodes and the first pair of electrodes. A portion of the elastically deformable between the first and second pairs of electrodes may define part of the inner surface of the first jaw.

In some cases, the first electrode may also be supported in the isolating portion. Thus, both the first electrode and the second pair of electrodes may be supported in the isolating portion.

The elastically deformable material may comprise silicone. Advantageously, silicone is a non-stick material, such that tissue and other biological matter may not stick to the isolating portion when silicone is used. This may avoid tissue sticking to the inner surface of the jaws. Silicone is also a good thermal insulator, which may contribute to low latent heat build-up in the instrument over the course of an electrosurgical procedure.

The isolating portion may interlock with one or more features on the second pair of electrodes. This may serve to ensure that the second pair of electrodes remains secured in the isolating portion. In this manner, the second pair of electrodes may be securely retained in the isolating portion, even when the isolating portion is deformed under compression. Such interlocking features may be particular beneficial where silicone is used, due to its non-stick nature.

Any suitable interlocking features which provide a mechanical connection (interlocking) between the isolating portion (elastically deformable material) and the second pair of electrodes for retaining the second pair of electrodes may be used. As an example, the third electrode and the fourth electrode may each comprise one or more through-holes into which the isolating portion protrudes, i.e. the one or more through holes may be filled with the elastically deformable material.

In some embodiments, the second pair of electrodes may comprise electrically conductive rails which are partially embedded in the elastically deformable material. In this manner, the second pair of electrodes may be supported in the elastically deformable material. The electrically conductive rails may be elongate elements of conductive material, which extend in a longitudinal direction of the jaws. The electrically conductive rails may have a greater rigidity than the elastically deformable material. In this manner, the elastically deformable material may deform in preference to the electrically conductive rails when pressure is applied to the second pair of electrodes.

In other embodiments, the electrosurgical instrument may further comprise an electrode support made from an electrically isolating material, wherein the third and fourth electrode each comprise a respective strip of conductive material on the support, and wherein the electrode support is partially embedded in the elastically deformable material. Thus, the second pair of electrodes may be provided on an electrode support, which is itself supported in the elastically deformable material. The strips of conductive material forming the third and fourth electrodes may be on a portion of the electrode support which protrudes from the elastically deformable material. The electrode support may be formed of a material which has a greater rigidity than the elastically deformable material, such that the elastically deformable material may deform in preference to the electrode support when pressure is applied to the electrode support. As an example, the electrode support may be made of a ceramic material, such as alumina.

The electrode support may include a support base plate and a support rib arranged on the support base plate to form a T-shape in a cross-sectional view of the jaws, wherein the third electrode and the fourth electrode each including a strip of electrically conductive material arranged on opposing sides of the support rib.

According to a second aspect of the invention, there is provided an electrosurgical apparatus for sealing and cutting tissue, comprising: a generator unit for generating radiofrequency and/or microwave electromagnetic energy; and the electrosurgical instrument according to the first aspect of the invention; wherein the transmission line is configured to convey the microwave energy to the first pair of electrodes and the radiofrequency energy to the second pair of electrodes. Any of the features described above in relation to the first aspect of the invention may be shared with the second aspect of the invention.

The generator unit may be configured to generate electromagnetic energy of a fixed single frequency or of a plurality of fixed single frequencies. Alternatively or additionally, the generator unit may be tuneable to generate electromagnetic energy of various frequencies, for example in a continuous range of frequencies between a minimum frequency and a maximum frequency. The generator unit may be connected to a power supply which provides the energy for generating the radiofrequency electromagnetic energy and/or microwave electromagnetic energy.

The generator unit is electrically and/or electronically (directly or indirectly) connected to the transmission line.

The generator unit may be configured to simultaneously generate microwave electromagnetic energy of a first frequency and radiofrequency electromagnetic energy of a second frequency. This may enable simultaneous tissue sealing with the microwave energy and RF cutting.

For example, the generator unit may include a generator that is configured to simultaneously generate electromagnetic energy of two different (fixed) frequencies. Alternatively, the generator unit may include a first generator for generating electromagnetic energy of the first frequency and a second generator for generating electromagnetic energy of the second frequency. The output of the first generator and output of the second generator can be combined using a multiplexer.

The multiplexer may be a diplexer and can combine the input from various sources into one output. For example, a multiplexer (or diplexer) is used to combine the output of first and second generators to a single output which is connected or coupled to the transmission line.

In an optional embodiment, a first coaxial cable is connected to the generator unit to receive the first frequency and a second coaxial cable is connected to the generator unit to receive the second frequency. Here again, the generator unit may include a first generator for generating electromagnetic energy of the first frequency and a second generator for generating electromagnetic energy of the second frequency. The first coaxial cable is connected to the first generator and the second coaxial cable is connected to the second generator.

The generator unit may be configured to alternatingly generate microwave electromagnetic energy of a first frequency and radiofrequency electromagnetic energy of a second frequency. For instance, the generator unit may include a first generator for generating electromagnetic energy of the first frequency, and a second generator for generating electromagnetic energy of the second frequency. The output of the first generator and the output of the second generator may be combined as described above using a multiplexer.

The generator unit may be configured to switch between generation of microwave energy and RF energy. Such a switching ability may be used to first seal the tissue using microwave energy and then subsequently cut the tissue using RF energy. Alternatively, the switch between the output of microwave energy and radiofrequency energy can be executed repeatedly and rapidly providing near simultaneous cutting and sealing.

Herein, the terms “proximal” and “distal” refer to the ends of the electrosurgical instrument, the jaws, the shaft and/or the coaxial transmission line further from and closer to a treatment site respectively. Thus, in use the proximal end is closer to a generator unit for providing the RF and/or microwave energy, whereas the distal end is closer to the treatment site, i.e. the patient.

The term “conductive” is used herein to mean electrically conductive, unless the context dictates otherwise. The terms “isolating” or “insulating” used herein may mean electrically isolating or insulating.

The term “longitudinal” used herein refers to a direction along the instrument channel parallel to the axis of the (coaxial) transmission line. In the context of the pair of jaws, the term “longitudinal” refers to the direction linking a proximal end of the jaws to a distal end of the jaws. Where the jaws are curved, the longitudinal “axis” may be considered as a line extending from a proximal end to a distal end of the jaws, and which is centred about a width of the jaws.

The term “lateral” refers to a direction that is perpendicular to the longitudinal direction. In the context of the jaw, the later direction may extend along a direction of a width of the jaws.

The term “inner” may mean radially closer to the centre (e.g. axis) of the instrument channel. The term “outer” may mean radially further from the centre (axis) of the instrument channel.

The term “electrosurgical” is used in relation to an instrument, apparatus or tool which is used during surgery and which utilises radiofrequency (RF) electromagnetic (EM) energy and/or microwave electromagnetic energy.

Herein, radiofrequency electromagnetic energy may mean a stable fixed frequency in a range 10 kHz to 300 MHz, preferably in a range from 100 kHz to 5 MHz, and more preferably in a range from 360 to 440 kHz. Microwave electromagnetic energy may mean electromagnetic energy having a stable fixed frequency in the range 300 MHz to 100 GHz. The radiofrequency electromagnetic energy should have a frequency high enough to prevent the energy from causing nerve stimulation. In use, the magnitude of the radiofrequency electromagnetic energy and the duration for which it is applied may be selected to prevent the energy from causing tissue blanching or unnecessary thermal margin or damage to the tissue structure. Preferred spot frequencies for the radiofrequency electromagnetic energy include any one or more of: 100 kHz, 250 kHz, 400 kHz, 500 kHz, 1 MHz, 5 MHz.

Preferred spot frequencies for the microwave electromagnetic energy include 915 MHz, 2.45 GHz, 5.8 GHZ, 14.5 GHZ, 24 GHz. 2.45 GHz and/or 5.8 GHz may be preferred.

### BRIEF DESCRIPTION OF THE DRAWINGS

Embodiments of the invention are described in detail below with reference to the accompanying drawings, in which:

FIG. 1 shows a schematic view of an embodiment of an electrosurgical apparatus;

FIG. 2a shows a schematic perspective view of a pair of jaws of an electrosurgical instrument according to an embodiment of the invention;

FIG. 2b shows a schematic part cut-away view of the jaws of FIG. 2a;

FIG. 2c shows a schematic cross-sectional view of the jaws of FIG. 2a;

FIG. 2d shows a schematic perspective view of a portion of a first jaw of the jaws of FIG. 2a;

FIG. 2e shows a schematic cross-sectional view of a pair of jaws of an electrosurgical instrument according to an embodiment of the invention

FIG. 3a shows a schematic perspective view of a pair of jaws of an electrosurgical instrument according to another embodiment of the invention; and

FIG. 3b shows a schematic part cut-away view of the jaws of FIG. 3a.

### DETAILED DESCRIPTION; FURTHER OPTIONS AND PREFERENCES

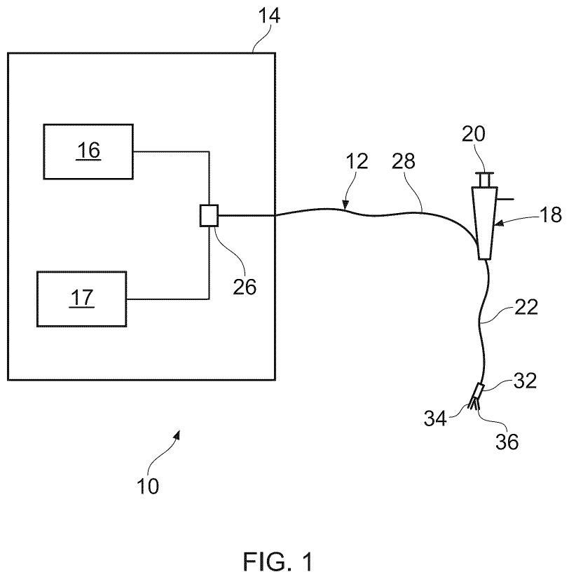

The present invention relates to an electrosurgical instrument and apparatus capable of delivering microwave energy to seal tissue (e.g. blood vessels) and/or of cutting the tissue. The electrosurgical instrument and apparatus may be used in open surgery but may find particular use in procedures where there is restricted access to the treatment site. For example, the electrosurgical instrument of the invention may be adapted to fit within the instrument channel of a surgical scoping device i.e. laparoscope, endoscope, or the like. FIG. 1 shows a schematic view of an electrosurgical apparatus 10 in which the electrosurgical instrument of the invention may be used.

FIG. 1 is a schematic diagram of an electrosurgical apparatus 10 that is an embodiment of the invention. The electrosurgical apparatus 10 is arranged to treat biological tissue using radiofrequency (RF) and/or microwave electromagnetic (EM) energy delivered from an electrosurgical instrument 12. The electromagnetic energy emitted by the electrosurgical instrument 12 into a treatment zone can be used to coagulate, cut, and/or ablate tissue in the treatment zone.

The electrosurgical apparatus 10 further comprises a generator unit 14 which can controllably supply radiofrequency and/or microwave electromagnetic energy to the electrosurgical instrument 12. The generator unit 14 may include a first generator 16 and a second generator 17. Suitable generators for this purpose are described in WO 2012/076844, which is incorporated herein by reference. The generator unit 14 may be arranged to monitor reflected signals received back from the electrosurgical instrument 12 in order to determine an appropriate power level for delivery. For example, the generator unit 14 may be arranged to calculate an impedance seen at the electrosurgical instrument 12 in order to determine an optimal delivery power level.

The electrosurgical apparatus 10 further comprises a surgical scoping device 18, such as a bronchoscope, endoscope, gastroscope, laparoscope or the like. The scoping device 18 may include a handpiece 20 and a flexible shaft 22. The handpiece 20 may include means for guiding the flexible shaft 22 through a cavity of a body. For example, the handpiece 20 can include means for moving a distal end of the flexible shaft 22 to change direction of the distal end of the flexible shaft 22. This helps manoeuvring the flexible shaft 22 through the cavity of the body. The flexible shaft 22 may include a working channel through which elongated structures can be moved and, thus, positioned at the treatment zone within the cavity of the body.

The first generator 16 and the second generator 17 are each configured to generate electromagnetic energy of a fixed frequency. However, the generator unit 14 is not limited to this configuration; the first generator 16 and/or the second generator 17 can be configured to generate AC electromagnetic energy in a continuous range between a minimum frequency and a maximum frequency. The frequency of the electromagnetic energy to be generated by the first generator 16 and/or the second generator 17 may be selected using an interface (not shown in the figures).

The generator unit 14 can include a combiner 26 which is configured to temporally switch between outputting the output of the first generator 16 or the output of the second generator 17. The combiner 26 may also be configured to combine the outputs of the first generator 16 and of the second generator 17. In this case, the combiner 26 acts as a multiplexer or diplexer.

The generator unit 14 is thus capable of generating and controlling power to be delivered to the electrosurgical instrument 12, e.g. via a transmission line 28, which extends from the generator unit 14 through the surgical scoping device 18 and instrument channel to the distal tip of the instrument channel. The generator unit 14 may have a user interface for selecting and/or controlling the power delivered to the electrosurgical instrument 12, e.g. controlling the first and/or the second generators 16, 17 and/or the combiner 26. The generator unit 14 may have a display for showing the selected energy delivery mode. In some examples, the generator unit 14 may allow for an energy delivery mode to be selected based on the size of the vessel to be sealed. Alternatively or additionally, the energy delivery is adapted based on tissue state.

The electrosurgical instrument 12 includes the transmission line 28, an instrument shaft (not shown in the figures), a joint 32, a first jaw 34, and a second jaw 36. Examples of the first and second jaws 32, 34 are discussed in more detail below. The transmission line 28 may include a coaxial cable that connects the generator unit 14 to the first jaw 34 and/or second jaw 34 for conveying the radiofrequency and/or microwave energy to the jaws, as discussed in more detail below.

The first jaw 34 and the second jaw 36 are movable relative to one another between an open position and a closed position. The first jaw 34 and the second jaw 36 are operably coupled to the joint 32 in a manner that enables opening and closing of the jaws, with the joint 32 being mounted on a distal end of the instrument shaft. The first jaw 34 and/or the second jaw 36 may pivotably mounted relative to the joint 32. The joint 32 may be arranged to ensure that the jaws remain laterally aligned as they are moved together. In some embodiments, the pair of jaws 34, 36 may comprise a static jaw that is fixed relative to the instrument shaft or the joint, with the other jaw being pivotable or rotatable. The joint 32 may include a pivot axle (not shown) which defines a pivot axis. The first jaw 34 and/or the second jaw 36 can pivot around the pivot axis or pivot axle. For example, the pivot axle may be fixed to the joint 32 and the first jaw 34 and the second jaw 36 can rotate around the pivot axle. Any other suitable mechanism for enabling relative movement between the jaws may be used.

A control wire or actuation rod which extends in the instrument shaft between the handle 20 and the joint 32 may be used for controlling opening and closing of the jaws. The joint 32 may comprise a converting mechanism which converts longitudinal back-and-forth movement of the control wire or actuation rod into a rotational movement of the first jaw 34 an/or second jaw 36, to enable opening and closing of the jaws.

In use, the first jaw 34 and the second jaw 36 are intended to grip biological tissue (in particular a blood vessel) therebetween. The first jaw 34 and the second jaw 36 are arranged to apply pressure to the biological tissue between opposing inner surfaces of the jaws 34, 36 and deliver energy (preferably microwave and/or radiofrequency electromagnetic energy) into the tissue from the transmission line 28.

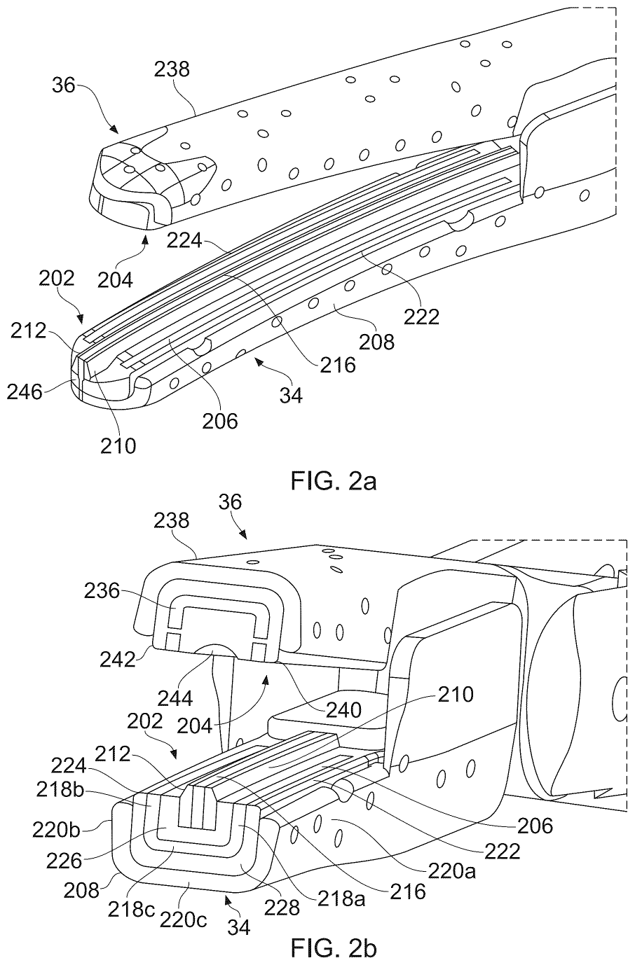

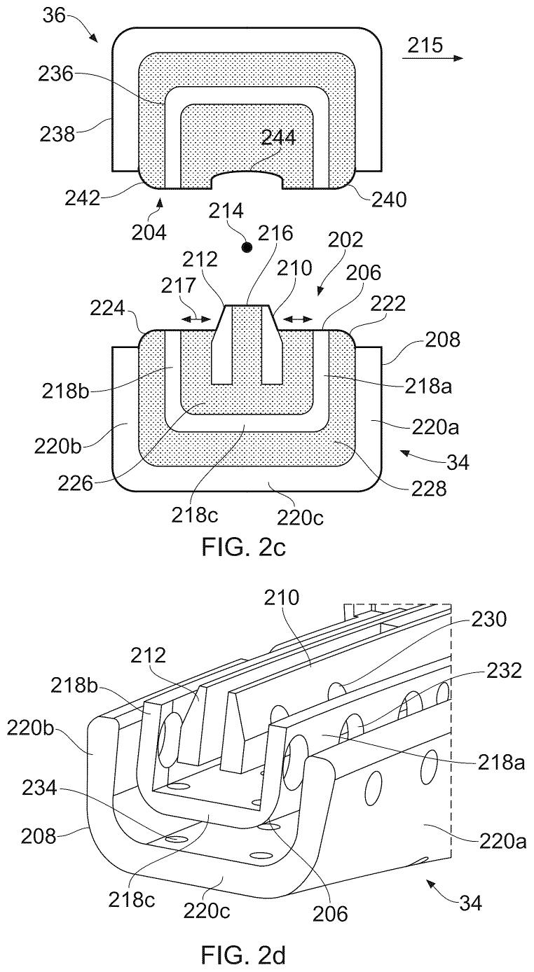

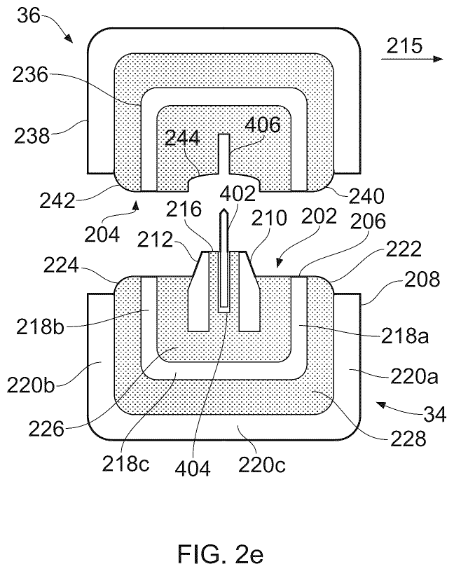

FIGS. 2a, 2b, 2c and 2d illustrate a first example of the jaws 34, 36 according to an embodiment of the invention. FIG. 2a shows a perspective view of the jaws 34, 36, whilst FIG. 2b shows a part cut-away view of the jaws 34, 36. A schematic cross-sectional view of the jaws 34, 36 is shown in FIG. 2c. The first jaw 34 includes a first inner surface 202 and the second jaw 36 includes a second inner surface 204, the first and second inner surfaces are exposed surfaces of the jaws which face towards one another. The first inner surface 202 and the second inner surface 204 can be brought into contact with one another by closing the jaws. In this manner, the first inner surface 202 and the second inner surface may be considered as pressure pads or pressure areas which can apply pressure to tissue located between the first jaw 34 and the second jaw 36.

The first jaw 34, shown as the lower jaw in the figures, comprises a first pair of electrodes for delivering microwave energy received from the transmission line 28, and a second pair of electrodes for delivering RF energy received from the transmission line 28. The first pair of electrodes comprises a first electrode 206 and a second electrode 208, whilst the second pair of electrodes comprises a third electrode 210 and a fourth electrode 212. The first pair of electrodes is connected to the transmission line 28 to receive microwave energy from the generator unit 14 conveyed by the transmission line 28, whilst the second pair of electrodes is connected to the transmission line 28 to receive RF energy from the generator unit 14 conveyed by the transmission line 28. In some embodiments, the transmission line 28 may comprise a first filter (e.g. an inductive filter) for blocking the microwave energy from reaching the second pair of electrodes, and a second filter (e.g. a capacitive filter) for blocking the RF energy from reaching the first pair of electrodes.

The third and fourth electrodes 210, 212 are in the form of a pair of conductive rails which are evenly spaced about a longitudinal axis 214 of the jaws and which extend parallel to the longitudinal axis 214. The longitudinal axis 214 of the jaws extends in a direction normal to a plane of the cross-sectional view of FIG. 2c, and extends from a proximal end of the jaws 34, 36 towards a distal end of the jaws 34, 36. The third and fourth electrodes 210, 212 are substantially evenly spaced about the longitudinal axis, so that the second pair of electrodes is centred with respect to a width of the first jaw 34. An RF cutting area 216 is defined on the first inner surface 202 between the third electrode 210 and the fourth electrode 212. In particular, tissue which is in contact with the RF cutting area 216 on the first jaw 34 may be cut when RF energy is conveyed to the second pair of electrodes via the transmission line 28.

The first electrode 206 defines a first channel in which the third and fourth electrodes 210, 212 are located. In more detail, the first electrode 206 has first and second side portions 218a, 218b which are located on either side of the second pair of electrodes, and which define sidewalls of the first channel. The first electrode 206 further includes a first connecting portion 218c, which connects the first and second side portions 218a, 218b and forms a bottom wall of the first channel. Thus, as shown in FIG. 2c, the first electrode 206 may have a U-shaped cross section which extends around part of the second pair of electrodes. The first and second side portions 218a, 218b of the first electrode 206 are substantially evenly spaced about the longitudinal axis 214 of the jaws. In this manner, a spacing between the first side portion 218a and the third electrode 210 may be the same as a spacing between the second side portion 218b and the fourth electrode 210.

The second electrode 208 is formed by a conductive outer shell of the first jaw 34. The conductive outer shell may be formed of a rigid conductive material, such as a metal or metal alloy. The second electrode 208 defines a second channel in which the first electrode 206 is located. In particular, the second electrode 208 has first and second side portions 220a, 220b which are located on either side of the first electrode 206, and which define sidewalls of the second channel. The second electrode 208 further includes a second connecting portion 220c, which connects the first and second side portions 220a, 220b and forms a bottom wall of the second channel. Thus, as shown in FIG. 2c, the second electrode 208 may have a U-shaped cross section, which extends around part of the first electrode 206. The first and second side portions 220a, 220b of the second electrode 208 are substantially evenly spaced about the longitudinal axis 214 of the jaws. In this manner, a spacing between the first side portions 218a, 220a of the first and second electrodes 206, 208 may be the same as a spacing between the second side portions 218b, 220b of the first and second electrodes 206, 208.

Thus, the second electrode 208 may form and outside of the first jaw 34, and define the second channel within which the first electrode 206 is located, the first electrode 206 itself defining the first channel in which the second pair of electrodes is located. A microwave sealing area is defined between the first electrode 206 and the second electrode 208 on the first inner surface 202. In particular, a first microwave sealing area 222 is defined on the first inner surface 202 between the first side portions 218a, 220a of the first and second electrodes 206, 208, whilst a second microwave sealing area 224 is defined on the first inner surface 202 between the second side portions 218b, 220b of the first and second electrodes 206, 208. Accordingly, the first and second microwave sealing portions 222, 224 are arranged on either side of the RF cutting area 216, and are substantially evenly spaced about the longitudinal axis 214. The first and second electrodes 206, 208 are arranged to radiate microwave energy received from the generator unit 14 into tissue which is in contact with the first and second microwave sealing areas 222, 224 on the first inner surface 202.

As can be seen, for example, in FIG. 2c, the side portions 218a, 218b of the first electrode 206 are spaced apart from the second pair of electrodes in a lateral direction normal to the longitudinal axis 214. As a result, the first and second microwave sealing areas 222, 224 are laterally spaced (offset) from the RF cutting area 216, such that there is no overlap between the RF cutting area 216 and the microwave sealing areas 222, 224 on the first inner surface 202. Accordingly, when a piece of tissue is clamped between the jaws 34, 36, microwave energy and RF energy may be applied to different portions of the tissue. This may avoid the portion of tissue which is in contact with the RF cutting area from being completely dried out by microwave energy delivered to tissue at the microwave sealing areas 222, 224. The lateral direction may be parallel to the inner surfaces 202, 204 of the jaws 34, 36, and is indicated in FIG. 2c by the arrow 215.

A minimum distance between the RF cutting area 216 and the microwave sealing areas 222, 224 may be set to ensure that tissue at the RF cutting area 216 is not excessively dried out due to application of microwave energy at the microwave sealing areas 222, 224. As an example, a minimum distance between the RF cutting area 216 and the microwave sealing areas 222, 224 may be at least 0.1 mm as this may be sufficient to avoid excessive drying out of tissue in the RF cutting area 216. In some cases, the minimum distance between the RF cutting area 216 and the microwave sealing areas 222, 224 may be at least 0.3 mm, 0.4 mm or 0.5 mm. The minimum distance in the lateral direction 215 between the RF cutting area 216 and the microwave sealing areas is indicated in FIG. 2c by the arrows labelled with reference numeral 217. As can be seen, the minimum distance (spacing) in the lateral direction 215 between the RF cutting area 216 and the microwave cutting areas 222, 224 corresponds to a spacing between the first side portion 218a of the first electrode and the third electrode 210 on one side, and to a spacing between the second side portion 218b of the first electrode and the fourth electrode 212 on the other side.

A spacing in the lateral direction 215 between the third and fourth electrodes 210, 212 may be at least 0.1 mm, and in some cases at least 0.3 mm. The spacing between the third and fourth electrodes 210, 212 may define a width of the RF cutting area 216. A spacing between the first electrode 206 and the second electrode 208 in the lateral direction 215 may be at least 0.1 mm, and in some cases at least 0.3 mm. In particular, the spacing between the first side portions 218a, 220a of the first and second electrodes may be at least 0.1 mm (or 0.3mm), and the spacing between the second side portions 218b, 220b of the first and second electrodes may be at least 0.1 mm (or 0.3 mm). Thus, the first and second microwave sealing areas 222, 224 may have a width of at least 0.1 mm (or 0.3 mm). Each of the jaws 34, 36 may have a width (in the lateral direction 215) between about 1.5 mm and 8 mm. As an example, the jaws 34, 36 may have a width of 3.45 mm.

The second pair of electrodes and the first electrode 206 are supported in the first jaw by a dielectric material. In more detail, the second pair of electrodes is supported in the first channel defined by the first electrode 206 by a first isolating portion 226 which is formed of a dielectric material. Thus, the second pair of electrodes is held in the first channel and spaced apart (isolated) from the first electrode 206 by the first isolating portion 226. Part of the first isolating portion 226 is exposed between the first electrode 206 and the second pair of electrodes, to form part of the first inner surface 202 of the first jaw 34. The first isolating portion 226 fills the first channel defined by the first electrode, and partially surrounds the second pair of electrodes, such that the third electrode 210 and the fourth electrode 212 are partially embedded in the first isolating portion 226. In this manner, portions of the third and fourth electrodes 210, 212 protrude from the first isolating portion 226, and thus protrude (project) from the first inner surface 202. The first isolating portion 226 also fills a gap between the third and fourth electrodes 210, 212, in order to isolate the third and fourth electrodes 210, 212 from one another.

The dielectric material of the first isolating portion 226 may be any suitable dielectric (insulating) material. In some embodiments, the dielectric material may be an elastically deformable dielectric material, such as silicone or the like. This may enable the second pair of electrodes to move relative to the first electrode 206 and/or other parts of the first jaw 34, in response to application of pressure to the second pair of electrodes. In particular, the second pair of electrodes may be supported in the first channel defined by the first electrode 206 only via the elastically deformable dielectric material. Thus, when the jaws 34, 36 are pressed together in the closed position, this may cause a pressure to be applied to the second pair of electrodes which results in elastic deformation of the first isolating portion so that the second pair of electrodes can move away from the second jaw 36. The second pair of electrodes (i.e. the third and fourth electrodes 210, 212) may have a higher rigidity compared to the elastically deformable dielectric material of the first isolating portion 226, such that the first isolating portion 226 may deform in preference to the second pair of electrodes when pressure is applied to the second pair of electrodes.

The first electrode 206 is supported in the second channel defined by the second electrode 208 by a second isolating portion 228 which is formed of a dielectric material. Thus, the first electrode 206 is held in the second channel and spaced apart (isolated) from the second electrode 208 by the second isolating portion 228. Part of the second isolating portion 228 is exposed between the first electrode 206 and the second electrode 208, to form part of the first inner surface 202 of the first jaw 34. In particular, the exposed portions of the second isolating portion 208 may correspond to the first and second microwave sealing areas 222, 224 discussed above. The second isolating portion 228 fills the second channel defined by the second electrode 208, and partially surrounds the first electrode 206. As shown in FIG. 2c, the first electrode 206 encapsulated between the first isolating portion 226 and the second isolating portion 228, such that the first electrode 206 is partially or completely embedded in dielectric material in the first jaw 34. In some cases, surfaces of the first electrode 206 on its first and second side portions 218a, 218b may be exposed at the first inner surface 202 of the first jaw 34. Alternatively, the first electrode 206 may be completely embedded in the dielectric material forming the first and second isolating portions 226, 228, such that none of its surfaces are exposed. The dielectric material of the first isolating portion 226 may be the same as the dielectric material of the second isolating portion 228. In some cases, the first isolating portion 226 may be continuous with the second isolating portion 228.

Similarly to the first isolating portion 226, the second isolating portion 228 may be formed of an elastically deformable dielectric material, such as silicone or the like. This may enable the first electrode 206 to move within the second channel, in response to pressure being applied to the first inner surface 202 of the first jaw 34. In particular, the first electrode 206 may be supported in the second channel only via the elastically deformable dielectric material of the second isolating portion 228. The first electrode 206 may have a higher rigidity compared to the elastically deformable dielectric material of the second isolating portion 228, such that the second isolating portion 228 may deform in preference to the first electrode 206 when pressure is applied to the first inner surface 202.

Flexible electrical connections, e.g. in the form of flexible wires, may be provided to connect the transmission line 28 to the first electrode 206 and the second pair of electrodes, to allow for movement of the electrodes within the jaw 34. For example, the joint 32 may include a set of flexible wires which electrically connects the transmission line 28 to the first electrode 206 and the second pair of electrodes.

The first isolating portion 226 may interlock with one or more features in the second pair of electrodes. This may ensure that the second pair of electrodes is securely held in the first isolating portion 226, which may be particularly beneficial where the first isolating portion 226 is formed of a non-stick material such as silicone. FIG. 2d shows a perspective view of the first jaw 34, where the first and second isolating portions 226, 228 are omitted, for illustration purposes. As can be seen, the third and fourth electrodes 210, 212 include a set of through-holes 230. The dielectric material of the first isolating portion 226 fills the through-holes 230 in the first and second electrodes 210, 212, to mechanically interlock the first isolating portion 226 with the third and fourth electrodes 210, 212.

Similarly, the first isolating portion 226 and/or the second isolating portion 228 may interlock with one or more features on the first electrode 206, which may ensure that the first electrode 206 is securely held within the first jaw 34. For example, as shown in FIG. 2d, the first electrode 206 includes through-holes 232 formed in its first and second side portions 218a, 218b. The dielectric material of the first and/or second isolating portions 226, 228 may extend into the through-holes 232 to provide mechanical interlocking with the first electrode 206. Preferably, the first and second isolating portions 226, 228 may be continuous with one another through the through-holes 232, such that the isolating portions 226, 228 form a continuous matrix in which the first electrode 206 and the second pair of electrodes are at least partially embedded. Where the first and second isolating portions 226, 228 are continuous with one another, they may be formed by pouring the dielectric material (in a fluid state) in the channels of the first and second electrodes 206, 208 while holding the first electrode 206 and the second pair of electrodes in their desired places, to fill the gaps between the electrodes. Additionally, the second electrode 208 (formed by the conductive outer shell of the first jaw 34) may include one or more features which interlock with the second isolating portion 228, to securely retain the second isolating portion 228 in the second channel defined by the second electrode 208. For example, the second electrode 208 may comprise a series of holes 234 (e.g. through-holes) into which the second isolating portion 228 extends, to provide mechanical interlocking with the second electrode 208.

As shown in FIGS. 2b and 2c, the second jaw 36 may comprise a third pair of electrodes including a fifth electrode 236 and a sixth electrode 238. The third pair of electrodes is configured to receive microwave energy conveyed by the transmission line 28, and to radiate the microwave energy into tissue located between the jaws 34, 36, e.g. to coagulate or seal the tissue. The structure of the third pair of electrodes is analogous to that of the first pair of electrodes (including the first and second electrodes 206, 208) in the first jaw 34 described above. In particular, the third pair of electrodes is arranged to mirror the first pair of electrodes, e.g. about a plane parallel to the inner surfaces of the jaws 34, 36. Thus, the third pair of electrodes defines a first microwave sealing area 240 on the inner surface 204 of the second jaw 36 between first side portions of the fourth and fifth electrodes 236, 238, and a second microwave sealing area 242 on the inner surface 204 of the second jaw 36 between second side portions of the fourth and fifth electrodes 236, 238. The microwave sealing areas 240, 242 on the second jaw 34 are arranged such that they are spaced apart from the RF cutting area 216 in the lateral direction 215, when the jaws 34, 36 are in the closed position. Thus, the microwave sealing areas 240, 242 on the second jaw 36 do not overlap the RF sealing area 216 in the lateral direction 215. The first and second microwave sealing areas 240, 242 on the second jaw 36 are arranged opposite the first and second microwave sealing areas 222, 224 on the first jaw 34, respectively. In this manner, when a piece of tissue is held between the jaws 34, 36, microwave sealing may be performed at corresponding locations on opposite sides of the piece of tissue, which may facilitate sealing the piece of tissue through its entire thickness.

The structure of the fifth electrode 236 is analogous to that of the first electrode 206 described above, with the fifth electrode 236 being arranged to mirror the first electrode 206. Thus, the fifth electrode 236, includes side portions and a connecting portion which define walls of a channel. Likewise, the structure of the sixth electrode 238 is analogous to that of the second electrode 208 described above, with the sixth electrode 238 being arranged to mirror the second electrode 208. Thus, the sixth electrode 238 is formed by a conductive outer shell of the second jaw 36, and includes side portions and a connecting portion which define walls of a channel in which the fifth electrode 236 is located. The channels in the fifth and sixth electrodes 236, 238 may be filled with a dielectric material, e.g. a same dielectric material as used in the first jaw 34 such as the elastically deformable dielectric material. Thus, the fifth electrode 236 may be supported in the second jaw 36 by a dielectric material in which it is at least partially embedded. The fifth and sixth electrodes 236, 238 may include one or more features (e.g. holes or through-holes) which interlock with the dielectric material, to provide a mechanical connection between the electrodes and dielectric material in the second jaw 36. The inner surface 204 of the second jaw 36 may further comprise a recess 244 which is arranged to receive at least part of the protruding second pair of electrodes when the jaws 34, 36 are in the closed position. The recess 244 may correspond to a channel which is formed in the dielectric material of the second jaw 36.

As shown in FIG. 2a, ends of the third electrode 210 and the fourth electrode 212 are exposed at a distal end of the first jaw 34. In particular, the ends of the third and fourth electrodes 210, 212 are exposed on a distal end face 246 of the first jaw 34, the distal end face 246 facing in a distal (forward direction). In this manner, the exposed ends of the third and fourth electrodes 210, 212 may act as a pair of RF electrodes on the distal end face 246 for cutting tissue located in front of the jaws.

This may enable tissue located in front of the jaws 34, 36 to be cut using RF energy delivered to the second pair of electrodes, which may, for example, enable the jaws 34, 36 to be inserted into (tunnelled through) target tissue. In some embodiments, the second jaw 36 may further include an overhang portion (not shown), which is arranged to cover the exposed ends of the third and fourth electrodes 210, 212 when the jaws are in the closed position. For example, the overhang portion may extend from a distal end of the second jaw 36 in a direction towards the first jaw 34 such that, when the jaws are in the closed position, the overhang portion is arranged in front of the ends of the third and fourth electrodes 210, 212. This may enable tissue to be gripped between the overhang portion on the second jaw 36 and the distal end face 246 of the first jaw 34, which may facilitate cutting tissue with the ends of the third and fourth electrodes 210, 212.

FIG. 2e shows a schematic cross-sectional view of a variation of the embodiment described above in relation to FIGS. 2a-2d. As shown in FIG. 2e, in some embodiments the first jaw 34 may further comprise a blade 402 for cutting tissue located between the jaws 34, 36. In particular, a slot 404 may be defined between the third electrode 210 and the fourth electrode 212, with the blade 402 being movable along the slot 404. For example, a dielectric carrier (element) may be located between the third and fourth electrodes 210, 212, the dielectric carrier defining the slot 404 along which the blade 402 is movable. Thus, the blade may be movable along a length of the RF cutting area 216, providing a further means for cutting tissue located in the RF cutting area. The slot 404 may extend along all or part of a length of the first jaw 34, such that the blade 402 may be movable (in the longitudinal direction) along all or part of the length of the first jaw 34. For example, the slot 404 may extend from a proximal end to a distal end of the first jaw 34. The second jaw 36 may further comprise a second slot 406, which is arranged to receive the blade 402 when the jaws 34, 36 are in the closed position. The slot 406 may extend along a length of the second jaw 36. The instrument 12 may comprise a control wire or an actuation rod for actuating the blade 402, i.e. for moving the blade 402 along the slot 404. The control wire or actuation rod may extend within the instrument shaft and be connected to the handle 20, which may be operable to control movement of the blade 402 along the slot 404. The blade 402 may be electrically isolated in the slot from the third and fourth electrodes 210, 212. Alternatively, the blade 402 may be electrically connected to one of the third and fourth electrodes 210, 212, to enable the blade 402 to act as an active or return electrode for the RF energy.

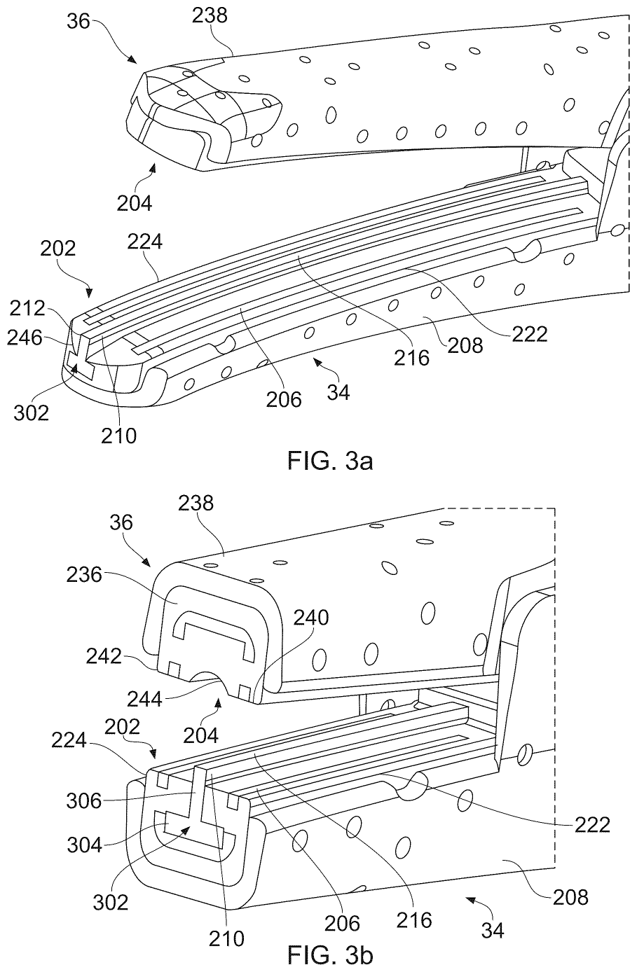

FIGS. 3a and 3b illustrate a second example of the jaws 34, 36 according to another embodiment of the invention. FIG. 3a shows a perspective view of the jaws 34, 36, whilst FIG. 3b shows a part cut-away view of the jaws 34, 36. The jaws 34, 36 of FIGS. 3a and 3b have a similar structure and operate in a similar manner to the jaws described in relation to FIGS. 2a-d discussed above. Accordingly, features of the jaws in FIGS. 3a and 3b which correspond to features of the jaws in FIGS. 2a-d are indicated with the same reference numerals as in FIGS. 2a-d, and are not described again.

In contrast to the embodiment of FIGS. 2a-d where the second pair of electrodes is formed as a pair of conductive rails which are floating in the first isolating portion 226, in the embodiment of FIGS. 3a and 3b, the second pair of electrodes is provided on an electrode support 302, which is formed of a rigid dielectric material. The electrode support 302 may, for example, be formed of a ceramic material, such as alumina. The electrode support 302 includes a support base plate 304 and a support rib 306 arranged on the support base plate, such that the electrode support has a T-shape in a cross-sectional view of the first jaw 34. The third electrode 210 and the fourth electrode 212 each include a strip made from an electrically conductive material and arranged on opposing sides of the support rib 306. In particular, the third and fourth electrodes 210, 212 are formed on a portion of the support rib 306 which protrudes from the inner surface 202 of the first jaw 34. Note than the fourth electrode 212 is not visible in FIGS. 3a and 3b, as it is located on a side of the support rib 306 which is not visible in the perspective views of the figures.

The support base plate 304 and/or the support rib 306 may each have an elongate shape and can be made from a plate or strip of electrically non-conductive material such as ceramic. The support base plate 304 can be (permanently) attached to the support rib 302, for example by adhesion. Alternatively, the support base plate and the support rib are a unitary component.

The electrode support 302 is centred within the first channel defined by the first electrode 206, and is mostly buried or covered by the first isolating portion 226, with only a portion of the support rib 306 being exposed at the first inner surface 202 or protruding from the first isolating portion 226. The support base plate 304 may be provided for interlocking the electrode support 304 with the first isolating portion 226. For example, the support base plate 304 protrudes on one or both sides from the support rib 306, such that a width of the support base plate 304 is larger than a thickness of the support rib 306, wherein the width and the thickness are measured in the lateral direction 215 of the jaws. The support base plate 304 may extend perpendicular to the support rib 306. The support base plate 304 may be attached to the support rib 306 at an end of the support rib 306 which is opposite to the end of the support rib 306 that is exposed at the first inner surface 202. The support base plate 304 may be fully embedded in the first isolating portion 226.

Where an elastically deformable dielectric material (e.g. silicone) is used for the first isolating portion 226, the dielectric material of the electrode support 302 may have a greater rigidity than the elastically deformable dielectric material. In this manner, the first isolating portion 226 may deform in preference to the electrode support 302 when pressure is applied to the electrode support 302, thus enabling the electrode support (and hence the second pair of electrodes) to move within the first jaw 34 in response to application of pressure.

The embodiment of FIGS. 3a and 3b may be adapted to include a blade (not shown), to provide a further means of cutting tissue between the jaws 34, 36. As an example, a slot may be defined within the support rib 306 of the electrode support 302, the slot extending in a longitudinal direction between the third and fourth electrodes 210, 212. The blade may then be located in the slot, and movable along the slot to cut tissue held between the jaws 34, 36. In particular, as the slot is located between the third and fourth electrodes 210, 212, the blade may serve to cut tissue along a length of the RF cutting area. In a similar manner to the example described above, the blade may be moved along the slot using a control wire or actuation rod which is connected to the handle 20.

1. An electrosurgical instrument for sealing or cutting tissue, comprising:
a transmission line for conveying microwave electromagnetic energy or radiofrequency electromagnetic energy;
a pair of jaws comprising a first jaw and a second jaw mounted at a distal end of the transmission line, wherein the first jaw and the second jaw are movable between an open position, in which tissue can be inserted into a gap between the first jaw and the second jaw, and a closed position, in which the first jaw and the second jaw are brought together to clamp tissue therebetween;
wherein the pair of jaws comprises a first pair of electrodes comprising a first electrode and a second electrode, and a second pair of electrodes comprising a third electrode and a fourth electrode;
wherein the first pair of electrodes is configured to receive microwave energy conveyed by the transmission line and to emit the received microwave energy into tissue located between the first jaw and the second jaw, such that a microwave sealing area is defined on an inner surface of the pair of jaws between the first and second electrodes;
wherein the second pair of electrodes is configured to receive radiofrequency energy conveyed by the transmission line and to operate as active and return electrodes for delivering the radiofrequency energy to tissue located between the first jaw and the second jaw, such that a radiofrequency cutting area is defined on an inner surface of the pair of jaws between the second and third electrodes; and
wherein the first pair of electrodes and the second pair of electrodes are arranged such that, when the first jaw and the second jaw are in the closed position, the microwave sealing area is spaced apart from the radiofrequency cutting area in a lateral direction normal to a longitudinal axis of the pair of jaws.

2. The electrosurgical instrument according to claim 1, wherein the radiofrequency cutting area is substantially centred relative to the longitudinal axis.

3. The electrosurgical instrument according to claim 1, wherein the first pair of electrodes is shaped to define a first microwave sealing area and a second microwave sealing area, and wherein the first and second microwave sealing areas are arranged on either side of the second pair of electrodes when the first jaw and the second jaw are in the closed position.

4. The electrosurgical instrument according to claim 3, wherein the first microwave sealing area and the second microwave sealing area are substantially evenly spaced about the radiofrequency cutting area in the lateral direction.

5. The electrosurgical instrument according to claim 3, wherein the first electrode comprises a first side portion and a second side portion which are spaced in the lateral direction by a first gap, and wherein the first and second side portions of the first electrode are located on either side of the second pair of electrodes when the first jaw and the second jaw are in the closed position.

6. The electrosurgical instrument according to claim 5, wherein the first electrode further comprises a first connecting portion which connects the first and second side portions of the first electrode across the first gap.

7. The electrosurgical instrument according to claim 5, wherein the second electrode comprises a first side portion and a second side portion which are spaced in the lateral direction by a second gap, and wherein the first electrode is located within the second gap.

8. The electrosurgical instrument according to claim 7, wherein the second electrode further comprises a second connecting portion which connects the first and second side portions of the second electrode across the second gap.

9. The electrosurgical instrument according to claim 7, when dependent on claim 4, wherein the first and second side portions of the second electrode are substantially evenly spaced about the longitudinal axis in the lateral direction.

10. The electrosurgical instrument according to claim 1, wherein the first jaw comprises the first pair of electrodes and the second pair of electrodes, and wherein the first pair of electrodes is spaced apart from the second pair of electrodes by a dielectric material in the first jaw.

11. The electrosurgical instrument according to claim 10, wherein:
the second jaw comprises a third pair of electrodes comprising a fifth electrode and a sixth electrode;
the third pair of electrodes is configured to receive microwave energy conveyed by the transmission line and to emit the received microwave energy into tissue located between the first jaw and the second jaw, such that a microwave sealing area is defined on an inner surface of the second jaw between the fifth and sixth electrodes; and
the third pair of electrodes is arranged such that, when the first jaw and the second jaw are in the closed position, the microwave sealing area on the inner surface of the second jaw is spaced apart from the radiofrequency cutting area in the lateral direction.

12. The electrosurgical instrument according to claim 1, wherein a minimum spacing in the lateral direction between the microwave sealing area and the radiofrequency cutting area is at least 0.1 mm.

13. The electrosurgical instrument according to claim 1, wherein the second pair of electrodes protrudes from an inner surface of a first one of the first and second jaws.

14. The electrosurgical instrument according to claim 13, wherein a second one of the first and second jaws comprises a recess for receiving a protruding portion of the second pair of electrodes when the first and second jaw are in the closed position.

15. The electrosurgical instrument according to claim 1, wherein ends of the third and fourth electrodes are exposed at a distal end of a first one of the first and second jaws.

16. The electrosurgical instrument according to claim 15, wherein a second one of the first and second jaws comprises an overhang portion arranged to cover the exposed ends of the third and fourth electrodes when the first and second jaws are in the closed position.

17. The electrosurgical instrument according to claim 1, wherein a slot is defined between the third electrode and the fourth electrode, and the instrument further comprising a blade which is movable along the slot to cut tissue located between the first jaw and the second jaw.

18. The electrosurgical instrument according to claim 1, wherein the second pair of electrodes is supported in one of the jaws by an isolating portion comprising an elastically deformable material, such that the second pair of electrodes is movable in the jaw upon application of pressure to the second pair of electrodes.

19. The electrosurgical instrument according to claim 18, wherein the elastically deformable material comprises silicone.

20. The electrosurgical instrument according to claim 18, wherein the isolating portion interlocks with one or more features on the second pair of electrodes.

21. The electrosurgical instrument according to claim 18, wherein the second pair of electrodes comprises electrically conductive rails which are partially embedded in the elastically deformable material.

22. The electrosurgical instrument according to claim 18, further comprising an electrode support made from an electrically isolating material, wherein the third and fourth electrode each comprise a respective strip of conductive material on the support, and wherein the electrode support is partially embedded in the elastically deformable material.

23. An electrosurgical apparatus for sealing and cutting tissue, comprising:
a generator unit for generating radiofrequency or microwave electromagnetic energy; and
the electrosurgical instrument according to claim 1;
wherein the transmission line is configured to convey the microwave energy to the first pair of electrodes and the radiofrequency energy to the second pair of electrodes.
